(19) United States
(12) Patent Application Publication
(10) Pub. No.: US 2018/0153467 A1
(43) Pub. Date: Jun. 7, 2018

(54) INJECTING AND MONITORING NERVOUS TISSUE

(71) Applicants: Yoav LICHTENSTEIN, Hod-HaSharon (IL); Yitzhack SCHWARTZ, Haifa (IL); Shlomo BEN-HAIM, Marlow (GB); Navix Internationl Limited, Tortola (VG)

(72) Inventors: Yoav LICHTENSTEIN, Hod-HaSharon (IL); Yitzhack SCHWARTZ, Haifa (IL); Shlomo BEN-HAIM, Marlow (GB)

(21) Appl. No.: 15/579,206

(22) PCT Filed: Jun. 2, 2016
(86) PCT No.: PCT/IB2016/053240

§ 371 Date: Dec. 3, 2017

Related U.S. Application Data
(60) Provisional application No. 62/170,266, filed on Jun. 3, 2015.

(51) Int. Cl. A61B 5/00; A61B 5/04; A61M 5/00; A61M 5/178; A61B 18/14; A61N 1/05

(52) U.S. Cl. CPC A61B 5/4848; A61B 5/6857; A61B 5/04001; A61B 5/4035; A61M 5/007; A61B 2018/00577; A61B 18/1492; A61N 1/0551; A61B 5/6882; A61M 2202/0484; A61M 5/178

(57) ABSTRACT

Sensing and/or treating GPs or other components of the ANS. Optionally, sensing is from within the GP, for example, using a helical needle with at least one electrode. Optionally, treatment is by injection of a neuromodulator chemical into the GP. In some embodiments, means are provided to reduce the migration of the neuromodulator away from the GP.

### RELATED APPLICATIONS

This application claims the benefit of priority under 35 USC § 119(e) of U.S. Provisional Patent Application No. 62/170,266 filed Jun. 3, 2015; the contents of which are incorporated herein by reference in their entirety.

This application is related to U.S. Provisional Patent Application No. 62/160,080; filed on May 12, 2015. The contents of the above application are incorporated by reference as if fully set forth herein in its entirety.

### FIELD AND BACKGROUND OF THE INVENTION

The present invention, in some embodiments thereof, relates to injecting nervous tissue such as ganglionic plexus and/or monitoring such tissue; and, more particularly, but not exclusively, to a catheter design for assisting in such injection and/or monitoring.

U.S. Patent Application No. 2007/0088244 discusses “Catheter-based systems are disclosed for geometrically and temporally controlled deliveries of fluid agents to the heart. Each system includes an elongate catheter shaft, a helical, linear or curved tissue penetration element at the distal end of the shaft, and a handle at the proximal end of the shaft for manipulating the penetrating element through the catheter shaft.

“The penetrating element and a conductive coil near the shaft distal end provide a pair of electrodes for bipolar sensing of tissue electrical activity. One version of the system includes a fluid lumen through the penetrating element and a contrast fluid lumen open at the catheter shaft distal end. In other versions of the catheter system, the penetrating element contains two fluid lumens. These systems facilitate a variety of tissue mapping and therapeutic agent delivery protocols in which several agents can be simultaneously delivered at a depth within heart tissue, prevented from intermingling until they reach the tissue. Treatment and contrast agents can be delivered simultaneously or temporally spaced, directed to the same region in tissue or to different regions separated by intervening tissue.”

An article entitled “Long-Term Suppression of Atrial Fibrillation by Botulinum Toxin Injection Into Epicardial Fat Pads in Patients Undergoing Cardiac Surgery”, published in Circulation: Arrhythmia and Electrophysiology. 2015; 8: 1334-1341 discusses:

“Animal studies demonstrated that autonomic hyperactivity and AF form a vicious cycle. The former can initiate AF and the latter can further enhance the autonomic neural activity.

“Suppression of the GP activity may be able to break this vicious cycle and suppress AF by prolonging the effective refractory period, reducing the dispersion of refractoriness, inhibiting triggered firing of both pulmonary veins (PV) and non-PV tissue as well as suppressing both the sympathetic and parasympathetic neural activity. It is also possible that reduction in early AF events facilitated reverse remodeling, which then helped diminish subsequent AF events. We hypothesize that these salutary effects led to the antiarrhythmic effects of botulinum toxin reported in the present study. In other words, by breaking the vicious cycle formed by AF and autonomic hyperactivity, the antiarrhythmic effects of botulinum toxin, which was expected to last for 2-3 months, extended to a year”.

Additional background art includes U.S. Application Publication Nos. 20120141532 and 20060182767; U.S. Pat. No. 8,744,571, International Patent Publication Nos. WO2006046065 and WO2014184746; and a paper titled “Catheter-based delivery of cells to the heart”, doi:10.1038/ncpcardio0446, in NATURE CLINICAL PRACTICE CARDIOVASCULAR MEDICINE SHERMAN ET AL. MARCH 2006 VOL 3 SUPPLEMENT 1.

### SUMMARY OF THE INVENTION

Following are some examples of some embodiments of the invention, also indicating some possible combination between various features of some embodiments of the invention.

### Example 1

A method of sensing the activity of nervous tissue of a patient, comprising: (a) advancing a part of a catheter including an electrode into nervous tissue of the Autonomous Nervous System (ANS) to be sensed; (b) modulating the activity of the nervous tissue while said catheter is in the body of said patient; and (c) sensing activity of said nervous tissue using said electrode before, during and/or after said modulating.

### Example 2

A method according to example 1, wherein said nervous tissue comprises a ganglionic plexus (GP).

### Example 3

A method according to example 1 or example 2, wherein said sensing comprises sensing before and sensing after said modulating.

### Example 4

A method according to any of the preceding examples, wherein modulating comprises directly interacting with said tissue.

### Example 5

A method according to any of the preceding examples, wherein modulating comprises interacting with an organ which the nervous tissue.

### Example 6

A method according to any of the preceding examples, wherein modulating comprises interacting with said body.

### Example 7

A method according to any of the preceding examples, wherein modulating comprises temporarily modulating.

### Example 8

A method according to any of the preceding examples, wherein modulating comprises electrical stimulation.

### Example 9

A method according to any of the preceding examples, wherein modulating comprises pharmaceutical stimulation.

### Example 10

A method according to any of the preceding examples, wherein modulating comprises modulating an activity of said nervous tissue with an effect expected to last at least 3 weeks.

### Example 11

A method according to example 10, wherein said modulating comprises ablating.

### Example 12

A method according to example 11, wherein ablating comprises ablating using said electrode.

### Example 13

A method according to example 10, wherein said modulating comprises injecting a chemical into said nervous tissue.

### Example 14

A method according to example 13, wherein said chemical comprises botulism toxin.

### Example 15

A method according to example 13 or example 14, wherein said chemical comprises an anti-inflammatory.

### Example 16

A method according to any of examples 13-15, wherein said chemical comprises a thickener.

### Example 17

A method according to any of examples 13-16, comprising a first injection to determine an expected spread of said chemical.

### Example 18

A method according to any of examples 13-17, comprising applying suction during or after said injection to reduce spreading of said chemical away from said nervous tissue.

### Example 19

A method according to any of examples 13-18, using a same fluid port for said chemical and for injecting a different material.

### Example 20

A method according to any of examples 13-18, using a different fluid port for said chemical and for injecting a different material.

### Example 21

A method according to any of examples 11-20, comprising also performing PVI.

### Example 22

A method according to example 21, wherein said PVI is performed using a same catheter as said catheter.

### Example 23

A method according to any of the preceding examples, comprising aiming said advanced part into a GP.

### Example 24

A method according to example 23, wherein aiming comprises verifying a location of said GP before said advancing.

### Example 25

A method according to example 23 or example 24, wherein aiming comprises navigating based on a plan indicating GPs.

### Example 26

A method according to any of examples 23-25, wherein aiming comprises verifying a depth of penetration.

### Example 27

A method according to any of examples 23-26, wherein aiming comprises checking a depth of penetration and/or penetration into a space.

### Example 28

A method according to any of examples 23-27, wherein aiming comprises selecting a spacer which sets a penetration depth of said part and attaching said spacer to a distal end of said catheter.

### Example 29

A method according to example 28, wherein said selecting comprises selecting based on an image of tissue in the vicinity of said nervous tissue.

### Example 30

A method according to any of the preceding examples, wherein said part extends from a distal end of said catheter and said distal end does not penetrate said nervous tissue.

### Example 31

A method according to example 30, wherein said extending part has a fixed length relative to said distal tip.

### Example 32

A method according to example 30, wherein said extending part is variably extendible form said distal tip.

### Example 33

A method according to any of examples 30-32, wherein said part is helical.

### Example 34

A method according to any of the preceding examples, wherein said sensing comprises bipolar sensing.

### Example 35

A method according to example 34, wherein two electrodes used for said bipolar sensing are advanced into said nervous tissue.

### Example 36

A method according to example 34 or example 35, wherein sensing comprises also sensing activity of an organ enervated by said nervous tissue.

### Example 37

A method according to example 36, wherein said sensing comprises measuring a change in conduction velocity.

### Example 38

A method according to example 36 or example 37, wherein said sensing comprises measuring a change in relationship in activity between two sections of nervous tissue.

### Example 39

A method according to any of the preceding examples, comprising repeating (a), (b) and (c) for a plurality of sections of nervous tissue during a same procedure.

### Example 40

A catheter, comprising: (a) a body having a distal end; (b) a needle extending from a distal end of said body; and (c) a spacer removably attachable to said distal end and limiting a maximal distance between a distal tip of said needle and said catheter.

### Example 41

A catheter according to example 40, wherein said needle is helical.

### Example 42

A catheter according to example 40 or example 41, wherein said needle includes at least one electrode thereon.

### Example 43

A catheter according to any of examples 40-42, wherein said spacer includes a channel for said needle.

### Example 44

A catheter according to any of examples 40-44, wherein said needle is selectively advanceable and retractable.

### Example 45

A catheter comprising: (a) a body having a distal end; and (b) a tissue penetrating element comprising a needle extending from said distal end; wherein (c) said tissue penetrating element comprises at least two electrodes, for bipolar measurement.

### Example 46

A catheter according to example 45, wherein said element comprises a single helical needle with two electrodes thereon.

### Example 47

A catheter according to example 46, wherein said helical needle includes at least one fluid outlet port.

### Example 48

A catheter according to example 46, wherein said helical needle includes at least two fluid outlet ports coupled to separate flow channels in said needle.

### Example 49

A catheter according to example 45, wherein said element comprises at least two helical needles.

### Example 50

A catheter according to example 45, wherein said distal end also includes an electrode.

### Example 51

A catheter according to example 45, wherein said element comprises at least two separate flow channels.

### Example 52

A catheter according to any of examples 45-51, wherein said catheter comprises at least two separate flow channels coupled to said needle.

### Example 53

A catheter according to any of examples 45-52, wherein a body of said catheter includes a contrast material flow channel terminating at an opening in said distal end.

### Example 54

A catheter according to any of examples 45-53, wherein said element is selectively retractable into said distal end.

### Example 55

A catheter comprising: (a) a body having a distal end; (b) a tissue penetrating element comprising a needle extending from said distal end, said needle including flow channel; and (c) a suction applying skirt at said distal end and coupled to a suction carrying channel in said catheter.

### Example 56

A system comprising: (a) a catheter having a needle mounted on a distal end thereof, said needle suitable for penetrating tissue and including fluid outlet port in said needle; and (b) a source of neuromodulating material coupled to said fluid outlet port.

### Example 57

A system according to example 56, comprising a source of contrast material coupled to said needle.

### Example 58

A system according to example 56 or example 57, comprising at least one electrode on said needle, coupled to at least one signal processor suitable for extracting and analyzing neural signals.

### Example 59

A system comprising: (a) a catheter having a needle mounted on a distal end thereof, said needle suitable for penetrating tissue and including a sensing electrode thereon; (b) at least one signal processor suitable for extracting and analyzing neural signals from said sensing electrode; and (c) a source of power for ablation coupled to said electrode in a manner suitable for ablating using said electrode.

### Example 60

A method of treating a GP, comprising:

injecting a chemical modulator of nervous activity into said GP; and

injecting an anti-inflammatory into said GP.

### Example 61

A method according to example 60, wherein said injecting and said injecting are carried out as a single injection of a mixture of said modulator and said anti-inflammatory.

### Example 62

A method according to any of examples 60-61, wherein said anti-inflammatory is a non-steroid anti-inflammatory.

### Example 63

A method according to any of examples 60-63, wherein said modulator is mixed with a thickener.

### Example 64

Use of a mixture of a neuromodulator and an anti-inflammatory to neuromodulate GPs.

### Example 65

Use of a mixture of a neuromodulator and a thickener to neuromodulate GPs.

### Example 66

A method of treating a GP, comprising: injecting a chemical modulator of nervous activity into said GP; and injecting a thickener into said GP.

### Example 67

A method according to example 66, wherein said thickener causes a carrier of a separately injected chemical modulator to thicken.

### Example 68

A method according to example 66, wherein said thickener is mixed with said chemical modulator.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the invention are herein described, by way of example only, with reference to the accompanying drawings. Some of the methods described herein are generally designed only for use by a computer, and may not be feasible or practical for performing purely manually, by a human expert. A human expert who wanted to manually perform similar tasks, such as GP signal processing, might be expected to use completely different methods, e.g., making use of expert knowledge and/or the pattern recognition capabilities of the human brain, which would be vastly more efficient than manually going through the steps of the methods described herein.

With specific reference now to the drawings in detail, it is stressed that the particulars shown are by way of example, and for purposes of illustrative discussion of embodiments of the invention. In this regard, the description taken with the drawings makes apparent to those skilled in the art how embodiments of the invention may be practiced.

In the drawings:

FIG. 1 is a flowchart of a method of ganglionic plexus (GP) sensing and/or treating, in accordance with some exemplary embodiments of the invention;

FIG. 2 is a schematic block diagram of a system for GP sensing and/or treating, in accordance with some exemplary embodiments of the invention;

FIG. 3 is a schematic showing of a tip design for GP sensing and/or treatment, in accordance with some exemplary embodiments of the invention;

FIG. 4 is a flowchart of a method of injection to a GP, in accordance with some exemplary embodiments of the invention;

FIGS. 5A-5C are a series of schematics showing stages of a method according to FIGS. 1 and 4, in accordance with some exemplary embodiments of the invention;

FIGS. 6A-6B show various alternatives for catheter tip design, in accordance with some exemplary embodiments of the invention;

FIG. 7 illustrates fluid flow in a catheter tip, in accordance with some exemplary embodiments of the invention;

FIGS. 8A-8C show depth sensing for a catheter tip, in accordance with some exemplary embodiments of the invention;

FIGS. 9A-9B show a fluid retention mechanism for a catheter tip, in accordance with some exemplary embodiments of the invention;

FIGS. 10A-10C show a depth control mechanism for a catheter tip, in accordance with some exemplary embodiments of the invention;

FIG. 11 is a flowchart of a method of signal processing, in accordance with some exemplary embodiments of the invention;

FIGS. 12A-12D schematically represent a sequence of operations for injection to a GP, according to some embodiments of the invention;

FIG. 13A represents a-D view of the atria of a heart reconstructed from CT data, according to some exemplary embodiments of the invention;

FIGS. 13B-13D show ¹²³I-mIBG NM images in the transverse, frontal (coronal), and medial (sagittal) planes, respectively, the NM images being fused with CT data; according to some exemplary embodiments of the invention; and

FIGS. 14A-14B show atria and in a lateral view, together with ganglia, according to some exemplary embodiments of the invention.

### DESCRIPTION OF SPECIFIC EMBODIMENTS OF THE INVENTION

The present invention, in some embodiments thereof, relates to injecting nervous tissue such as ganglionic plexus and/or monitoring such tissue; and, more particularly, but not exclusively, to a catheter design for assisting in such injection and/or monitoring.

### Overview

A broad aspect of some embodiments of the invention relates to measuring and/or modulating parts of the autonomic nervous system (ANS) as part of diagnosis or treatment, for example. In some exemplary embodiments of the invention, the ANS is considered as including nervous tissue, including for example, efferent nerves and afferent nerves and sympathetic and parasympathetic nerves. In some cases parts of the ANS include synapses, for example, concentrated in ganglionic plexi (GP), possibly arranged as a network. The ANS is believed to have a role in controlling organ and/or system function, for example, by modifying an excitability level of tissue and/or a response to stimulation of tissue. It is also believed that some organ and tissue disorders manifest themselves and/or can be treated by modifying parts of the ANS.

In some exemplary embodiments of the invention, parts of the ANS, for example, GPs are sensed to help diagnose disorders and/or are modulated to help treat disorders. While the application in general includes description of treating GPs, in some embodiments of the invention other parts of the ANS, for example, nerve fibers are treated instead or in addition. Also, while the application in general uses examples of the heart, methods and apparatuses described herein may be used for other organs; for example, hollow organs such as the stomach and intestines, and solid organs such as the liver and brain. Optionally, access is via lumens of the organ and/or lumens of nearby blood vessels. It is noted that the heart may be an especially difficult organ to treat due to its movement, complex control structure and/or criticality in the body. In some exemplary embodiments of the invention, the GPs treated include small GPs, for example, less than 7 mm, 5 mm, 3 mm, 2 mm, 1 mm or intermediate maximal extents.

In some embodiments, the GPs are located near and/or accessed through easily damaged tissue, such as major blood vessels or damaged or thin atrial wall. In some embodiments, nerves, for example, with a diameter of between 0.5-2 mm, are treated.

Optionally, any injection is adjacent to the nerve. Alternatively, injection is to the nerve. In some embodiments, end effectors are treated.

An aspect of some embodiments of the invention relates to sensing GP activity using an electrode mounted on a needle positioned in or near the GP; and delivered, for example, using a catheter. Optionally, activity sensing is used before, during and/or after treatment. In some exemplary embodiments of the invention, treatment comprises injection of one or more drugs which affect the ANS. Optionally or alternatively, treatment comprises electrical and/or other ablation. In some exemplary embodiments of the invention, treatments is used to modify the behavior of an organ, such as a heart or stomach; and sensing is used in addition to or instead of sensing of organ behavior and/or as a means for predicting the effect on organ behavior. In some exemplary embodiments of the invention, the sensing and/or feedback from the organ includes real time sensing and/or feedback. In some embodiments, this may allow better control over the treatment of the GP (e.g., to achieve a desired effect on the GP). Optionally or alternatively, in some embodiments this may allow modification of the GP treatment in order to achieve a desired effect on the organ.

In some exemplary embodiments of the invention, sensing is bipolar using two electrodes in or near the GP. Optionally, sensing is unipolar using one electrode in the GP and an optional far electrode; for example a body surface electrode or an electrode of a distance at least 5 cm, 10 cm, 15 cm, 30 cm, or another distances in cm away from the GP; e.g., along the catheter.

In some exemplary embodiments of the invention, the treatment comprises treating multiple GPS and/or a GP modification and/or a different treatment, for example, pulmonary vein isolation (PVI) or rotor ablation. Optionally, using methods and/or apparatus and/or treatments as described in U.S. Ser. No. 62/160,080, IB2015/050148, WO2015/033317, WO2015/033319 and/or WO2014/115151, may be followed.

In some exemplary embodiments of the invention, a same electrode is used for ablation and for sensing of GP tissue. Optionally or alternatively, a same electrode is used for sensing electrical activity and for impedance measurement (e.g., for tissue characterization). Optionally or alternatively, a same electrode is used for sensing GP tissue activity and for stimulating and/or ablating GP tissue.

In some exemplary embodiments of the invention, the sensing electrode is selected to have a low impedance and/or large surface area (e.g., achievable by coating and/or surface geometry). Optionally, changes in impedance are provided using circuitry, rather than the electrode design. In some cases a same electrode is used for power delivery and sensing and the actual electrode size and/or properties are a tradeoff. In some embodiments, sensing electrodes are optimized with respect to size, area, coating and/or impedance for sensing.

Optionally or alternatively, at least one electrode is not shared between sensing GP tissue activity and other uses.

An aspect of some embodiments of the invention relates to a needle configuration for GP treatment. In some exemplary embodiments of the invention, the needle is adapted to anchor in or near a GP and includes at least one electrode for measuring activity from the GP.

In some exemplary embodiments of the invention, two electrodes are provided on the needle. Optionally or alternatively, two needles are provided. Optionally or alternatively, one electrode is provided on the needle and one near its base.

In some exemplary embodiments of the invention, the needle includes at least one fluid port for injecting a fluid into the GP. Optionally, the fluid is a contrast material, used, for example, to verify the needle location. Optionally or alternatively, the fluid is a chemical ablation fluid. Optionally or alternatively, the fluid is a marker.

A potential advantage of an injection modality for ablation is reduction of inadvertent collateral damage (e.g., impairment of nervous activity can potentially be obtained without requiring wholesale destruction of tissue). In particular, there is a potential reduction of risks to the patient when target tissue is located near critical and/or vulnerable structures such as aorta, esophagus, and/or phrenic nerve. In the case of epicardial GPs that are embedded in fat pads, injection, optionally even injection of tissue-destroying material, is potentially more effective than certain ablation procedures such as RF ablation, which may not work as well inside fatty tissue.

Optionally or alternatively, the fluid is a pharmaceutical, for example, botulinum toxin, which has a temporary (e.g., 1-1000 seconds, 1-40 days, 1-10 weeks or intermediate or longer periods) effect (e.g., blocking) on nerve tissue. A potential advantage of a fluid having temporary effects on nerve tissue is the capacity to act as a “reset” on hyperactive autonomic nervous control of the heart, without the requirement of permanent denervation. For example, botulinum toxin potentially numbs GPs locally and temporarily without physically disrupting the GP or nerve endings. As a temporary activity blocking effect recedes, the GPs potentially regain activity and the autonomic control of the heart returns. Rearrangements of tissue and/or control during recovery potentially restore a more normal pattern of activity than was present before the treatment. In some embodiments, the same and/or different GP are re-treated by one or more subsequent rounds of injection, which has the potential advantage of allowing treatment to be tuned on the basis of previous treatment results.

In some embodiments, particularly where replication and/or controlled variation of previous results is targeted, it is a potential advantage to be able to reproducibly replicate aspects of the injection protocol such as injection depth. In some embodiments, injection depth is controlled and/or monitored through the mechanical configuration of the injection device and/or according to how it is controlled (e.g., according to the size of a spacer, and/or a number of helical turns used to advance).

An aspect of some embodiments of the invention relates to sensing GP activity using an electrode mounted on a needle positioned in or near the GP and delivered, for example, using a catheter. Optionally, activity sensing is used before, during and/or after treatment. In some exemplary embodiments of the invention, treatment comprises injection of one or more drugs which affect the ANS. Optionally or alternatively, treatment comprises electrical and/or other ablation.

An aspect of some embodiments of the invention relates to neural conduction velocity measurements between a GP and other tissue, such as the AV node.

Optionally, this is used to assess a degree of connectedness of two neural tissues and/or an effect of modification of a GP on conduction. In some exemplary embodiments of the invention, measurement is between an electrode in a GP and an electrode on an endocardial surface or in another GP.

An aspect of some embodiments of the invention relates to injecting combinations of pharmaceuticals into GP(s), including, for example, both a nerve activity modifying agent, such as botulinum toxin and a repair related agent, such as an anti-inflammatory, such as an NSA.

An aspect of some embodiments of the invention relates to a needle electrode suitable for anchoring in a GP. In some exemplary embodiments of the invention, the needle electrode is in the form of a helix. Optionally, the helix includes at least two electrodes which can be used for bipolar sensing there between.

It is noted that some GPs to be treated in accordance with some embodiments of the invention are between 1 and 15 mm (e.g., 3-10 mm) in maximal extent and/or between 1 and 10 mm (e.g., 2-7 mm) in minimal extent. In an exemplary embodiment of the invention, the helix used is small in diameter enough to fit in such a GP and/or uses a thin enough body to avid over-damaging such a small GP. Optionally or alternatively, the helix is short enough so it does not penetrate too far past the GP, but long enough to reach from inside the heart via an atrial wall, to the GP. Optionally or alternatively, a plurality of electrodes and/or fluid ports are provided so at least one or two of the electrodes and/or fluid ports are correctly located.

In some exemplary embodiments of the invention, when in use, the needles are used gently enough to prevent atrial wall tearing. Optionally or alternatively, navigation methods, for example as described herein, and/or ultrasonic imaging (optionally on a guidewire or stylet within the catheter) are used to avoid important structures such as blood vessels.

In some embodiments of the invention, a plurality of helical needles or a helical needle and a non-helical needle are provided.

An aspect of some embodiments of the invention relates to a depth control for a needle. In an exemplary embodiment of the invention, a catheter includes a kit with one or more spacer caps, each with a different (e.g., predefined) thickness, which can be mounted at a tip of the catheter and partially covering a needle electrode extending from the catheter. Optionally, the needle is a helical needle. In an alternative embodiment of the invention, a rod or tube is carried by the catheter, either within the helix or surrounding the helix and can be selectively advanced (and optionally locked in place) to control insertion depth of the helix.

In some exemplary embodiments of the invention, a desired advancement depth is determined before or during the procedure, for example using imaging, for example, ultrasonic imaging from the catheter tip or CT or MRI imaging (e.g., pre-operative CT or MRI imaging). Optionally, a spacer is used to preset a maximum needle insertion depth. Optionally, the preset is to a range of 0-5 mm, for example, with a depth precision (of insertion) of 2 or 1 mm or better.

An aspect of some embodiments of the invention relates to selective sensing form a GP. In some exemplary embodiments of the invention, this is provided by using two electrodes within or near (e.g., 1-3 mm) of the GP. Optionally or alternatively, one electrode is mounted on a catheter tip (e.g., a base of a needle electrode) and an extending needle electrode acts as or has mounted thereon a second electrode.

An aspect of some embodiments of the invention relates to position determination of a GP treatment device. In an exemplary embodiment of the invention, such determination comprises injecting contrast material from the tip of the device (or otherwise near an active part thereof) and imaging spread of such material.

Optionally or alternatively, optical sensing, for example of a degree of reflectivity of surrounding tissue, is used to detect if the needle is in tissue or in or near a space, such as a pericardial space. Optionally or alternatively, impedance measurements are used to characterize the tissue (e.g., as being neural tissue), using electrodes on the needle.

An aspect of some embodiments of the invention relates to chemical treatment of a GP. In some exemplary embodiments of the invention, the chemicals are injected using a needle directly to a GP, while the needle is anchored to a wall of a nearby lumen, such as a cardiac muscle.

An aspect of some embodiments of the invention relates to determining a scope of effect of a range of treatment of an injection-type treatment. In some exemplary embodiments of the invention, the scope is predetermined by injecting a viscous or otherwise modified material, for example including a toxin, for example as described in Toxins 2011, 3:63-81; doi:10.3390/toxins3010063, which increases local retention, reduces spread of the material and/or prevents backwash into the blood and potentially toxic local and/or systematic side-effects. Optionally or alternatively, the scope is predetermined by injecting contrast material and determining an expected spread of later injected material, possibly modifying one or more injection parameters before injection. Optionally or alternatively, the scope is controlled by applying suction to a location away from where spread is suspected. Optionally or alternatively, scope is controlled by the positioning of fluid outlets of an injector at a side of the injector.

An aspect of some embodiments of the invention relates to injecting high-viscosity fluid via a small diameter channel in a needle, especially if the needle is used for injecting multiple fluids. In some exemplary embodiments of the invention, a single channel is provided in the needle and shared by materials arriving from multiple fluid channels in a catheter carrying the needle. Optionally, a valve is used to prevent cross-contamination between the channels.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

Exemplary Sensing and/or Treating

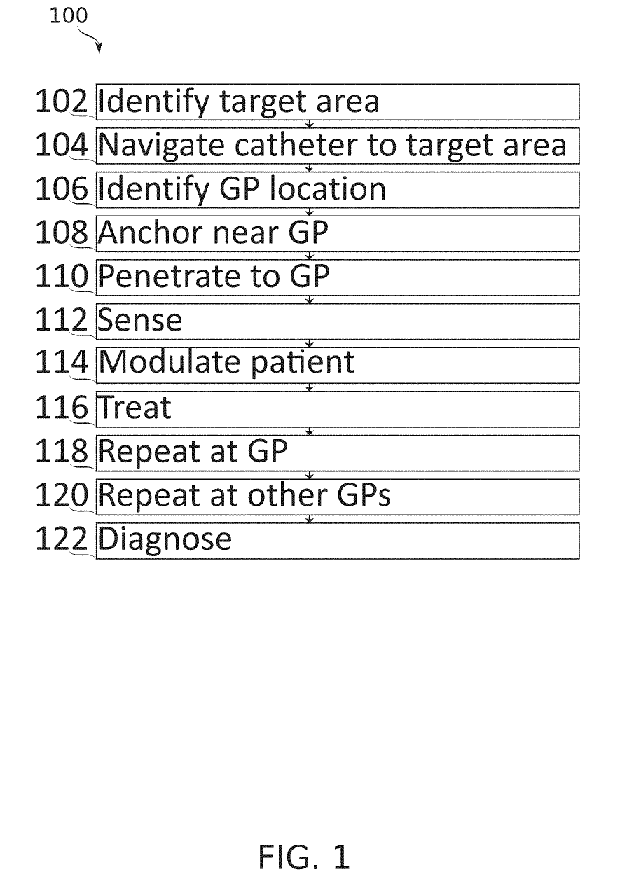

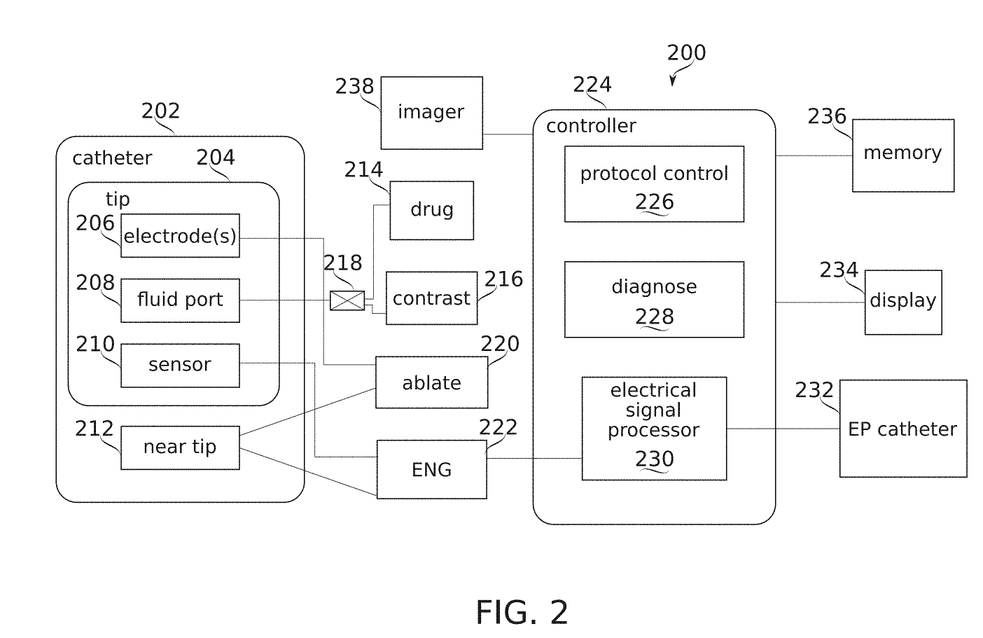

An exemplary method of sensing and/or treating GPs is described with reference to FIGS. 1 and 2. FIG. 1 is a flowchart 100 of a method of ganglionic plexus (GP) sensing and/or treating, in accordance with some exemplary embodiments of the invention.

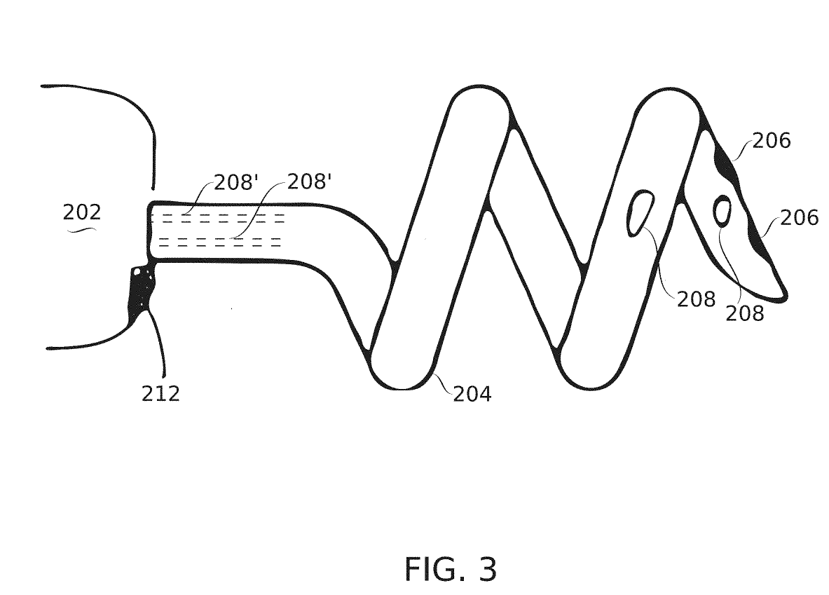

FIG. 2 is a schematic block diagram of a system 200 for GP sensing and/or treating, in accordance with some exemplary embodiments of the invention. In an exemplary embodiment of the invention, system 200 includes one or more catheters 202, each with one or more tips 204 adapted for insertion into solid tissue, optionally a GP. As also described for example in relation to FIG. 3 herein; tip 204 may include one or more electrodes 206; for example, for sensing and/or ablation. Optionally or alternatively, tip 204 includes one or more fluid ports 208. Optionally, one or more sensors 210 are provided in tip 204. Optionally, a near-tip element 212, such as an electrode or a spacer, is provided. Optionally or alternatively, other tip features are provided; for example a port for contrast material (e.g., for use during navigation), a guidewire port and/or an ultrasonic imager.

System 200 optionally also includes a catheter controller 224, optionally with memory 236 (e.g., for images) and/or a display and/or other UI components 234. It is noted that, in some embodiments, some components of system 200 may be controlled directly by a user. Controller 224 optionally includes one or more of a protocol control 226, for example, to manage an ablation (or other treatment) and/or sensing protocol. For example, protocol control 226 (and/or a manual input) may be used to control injection of a drug from a source 214 and/or contrast material from a source 216; optionally using to a fluid controller or valve 218 to control delivery of such fluid to a fluid port 208. Optionally or alternatively, an optional ablation system 220 is controlled by protocol controller 226. Optionally or alternatively, a diagnosis module 228 is provided, for example, to diagnose an organ state and/or a GP state based on GP measurements and/or other measurements. Additional data may be provided from outside systems for controller 224, for example, from an imager 238.

An electrical signal processor 230 is optionally provided, for example, to analyze data sensed from an electroneurography (ENG) circuit 222 and/or data from an EP catheter 232.

Referring back to FIG. 1, an exemplary process of GP sensing and/or treatment optionally starts with identifying a target area at block 102, for example, a fat pad or a suspected GP location. Optionally, the identification is based on a measurement or an estimation of a location of a malfunctioning or otherwise treatable GP. In one example, nuclear medicine (NM) imaging, for example as described in one of the above mentioned PCT applications and publications, is used to identify GPs and a diagnosis process may be used to assess which GPs it may be desirable to treat. Optionally, actually treating such GPs, more precise measurements of their activity and/or determining if treating such GPs is a correct treatment for the patient, optionally use system 200, for example, as described below.

At block 104, the catheter is navigated to the target location. Optionally, standard intra-luminal navigation methods are used, for example, one or more of position sensors, specially shaped guide wires, stylets and/or guide catheters. In some embodiments, a laparoscopic approach, for example, using a rigid or a flexible tool is used. In some embodiments and/or for some targets, the catheter is replaced by a needle which is advanced to the target area. Optionally, navigation is under imaging (e.g., angiography) and/or in reference to a previously acquired image and/or anatomical map captured using, for example, a position sensor.

At block 106, the GP location (or location of other nervous tissue of interest) is optionally identified. In some exemplary embodiments of the invention, GP location (and/or a location of cardiac muscle or luminal wall nearest such GP and/or through which a GP can be accessed from the lumen) is identified based on anatomical considerations, for example, it being in a fat pad. Optionally or alternatively, position of the GP is identified on an image and the position of the catheter (or other probe) is registered to that image. Optionally or alternatively, GP location is based on measurement of nerve signals, for example, from a lumen wall near the GP. In an exemplary embodiment of the invention, GP location is determined by applying high frequency stimulation (HFS) and identifying an effect of such stimulation on the organ. It is generally accepted that HFS of a GP has a clear effect on the heart rate and/or systemic blood pressure. Similarly, such stimulation may be used in the stomach and then detecting changes in the activity of the stomach based on stimulation of the HFS.

At block 108, the catheter is optionally anchored near the GP. In an exemplary embodiment of the invention, the anchoring is by using a helical tip 204 for the catheter which is screwed into the nearby tissue (e.g., as described in relation to FIG. 3) or using a barb or movable jaws at the tip of the catheter and/or by other anchoring means, such as suction.

In some exemplary embodiments of the invention, anchoring is important to ensure that the catheter does not move during treatment and/or measurement and/or between positioning and treatment.

At block 110, tip 204 is optionally penetrated to the GP; optionally into the GP.

Optionally, the penetration is by further rotation of a helical tip design.

Optionally or alternatively, penetration is by advancing of a generally linearly moving needle.

Location in the GP is optionally confirmed by one or more of: position sensing (e.g., relative to a map, for example, using position sensing), impedance measurements, estimation (e.g., based on an expected depth) and/or using injection of contrast material and estimating position based on dispersion thereof. In some embodiments, what is determined is not the location relative to a GP, but rather relative to other tissue, for example, relative to a pericardium. Optionally, for example, as described below, this is determined using an optical sensor.

At block 112, signals from the GP are optionally sensed. In an exemplary embodiment of the invention, sensing is bipolar, for example, between two electrodes 206 on tip 204 (e.g., inside the GP) or between the tip and a near-tip electrode 212. Such bipolar sensing may also be used to detect the GP and/or to confirm that tip 204 is placed near enough to or in the GP.

In some exemplary embodiments of the invention, sensing is used to provide an initial diagnosis of the GP, for example, to determine its health state and/or to determine a base line thereof.

In some embodiments, GP measurements are collected over time, for example, to detect natural variations. In some embodiments, the patient is actively modulated at block 114 (e.g., stimulated using electrical, mechanical or chemical means) in a manner expected to cause a change in GP activity, and this activity is optionally measured and/or correlated with the modulation. In some exemplary embodiments of the invention, the patient is modulated by modulating the GP, for example, by stimulating it or inhibiting its activity (e.g., using electricity or suitable chemicals). In some exemplary embodiments of the invention, the stimulation is local, for example, electrically stimulating the GP or nearby tissue and/or stimulating the organ as a whole (e.g., distending thereof). Optionally or alternatively, the stimulation is global, for example by causing pain or physiological stress.

Optionally or alternatively to collecting GP signals, other physiological data may be collected, for example, regarding the activity of the organ controlled by the GP and/or other body system.

At block 116, the patient is optionally treated, for example, by ablating part or all of the GP or by injection of a material thereinto. Optionally, material injection is via one or more ports 208. Optionally or alternatively, electrical-based ablation is between electrodes 206 and/or between electrode(s) 206 and a near-tip electrode 212.

In some embodiments, treatment comprises a temporary treatment and then a more permanent treatment. For example, first a material with a short term effect is injected, for example, cold saline, and then a toxin is injected (or a different modulation type, for example, needle ablation), for example, after an assessment of tissue response to the temporary treatment. In another embodiment, the more permanent effect (e.g., which lasts a plurality of days, weeks and/or months, is delayed. In one example, gold nanoparticles are injected. In another embodiment microspheres which absorb ultrasound energy and/or which can open to release a bioactive chemical, are injected. The treatment is optionally effected at a later time by activating the microspheres and/or nanoparticles.

Any or all of blocks 112-116 are optionally repeated at the GP (at block 118), optionally after a delay (e.g., 1-120 seconds, minutes and/or between 1 and 3 hours); for example, to allow the GP and/or organ and/or other body system to return to a baseline condition.

Any or all of blocks 102-118 are optionally repeated at another GP (at block 120). In some embodiments, a plurality of GPs (e.g., 2, 3, 4 or more) are treated and/or modulated and/or stimulated substantially simultaneously (e.g., either at same time or with overlapping effects). In some embodiments, a region known to have several GPs, for example a fat pad, is treated as a whole or in part, potentially simultaneously affecting multiple GPs directly.

At block 122, the patient is optionally diagnosed, based on the reactivity of one or more GP and/or one or more body organs.

### Exemplary Catheter Tip Design

FIG. 3 is a schematic showing of a tip design 204 for GP sensing and/or treatment, in accordance with some exemplary embodiments of the invention. As can be seen tip 204 is in a general helical form and includes one or more electrodes 206 and/or one or more fluid ports 208 nears its distal end.

In some exemplary embodiments of the invention, for example, as shown, the helix connects to a tip of catheter 202 at a near tip area 212 (which may include an electrode). Optionally, the connection includes an abrupt change in diameter, optionally selected to prevent over insertion of tip 204 by preventing advance of near tip area 212 into tissue. In some embodiments, the helix is extendible from a lumen in catheter 202. Optionally or alternatively, the helix may be fixed in place, optionally with a retractable over tube being provided.

As shown, two electrodes 206 are shown within, for example, 1 mm from the distal end of tip 204. One or more electrodes may be at various locations along the tip, for example, in a first axial third, a second axial third, and/or a most distal third. Any two electrodes may be near each other (e.g., within 20% of an axial length of the tip) or more distant from each other. Optionally or alternatively, more electrodes may be provided, for example, 3, 4, 5 or more.

In some exemplary embodiments of the invention, one or more electrode 206 is a patch electrode, for example, facing an inside of the helix or away from the helix. Optionally or alternatively, one or more of the electrodes is a ring electrode, surrounding to the tip. In some embodiments, multiple electrodes facing in different directions are electrically interconnected to form a single electrode.

In some embodiments tip 204 itself acts as an electrode.

Tip 204 may include one or more fluid channels 208′ which terminate at one or more fluid outlets 208. Optionally, the fluid outlets are in a distal or middle third of the axial length of tip 204. In some embodiments, a port 208 is formed at the distal end of tip 204 (e.g., tip is hollow at its end), pointing forward. In some exemplary embodiments of the invention, a port 208 faces (sideways) inwards. This may help aim the dispersion of injected material. Optionally or alternatively, a port 208 faces outwards.

In some exemplary embodiments of the invention, a helical tip 204 has a length of, for example, between 1 and 50 mm; for example, between 2 mm and 5 or 10 mm. Optionally or alternatively, tip 204 has a helical diameter of between 0.3 and 4 mm, for example, between 1 and 2.1 mm. Optionally or alternatively, tip 204 has a diameter smaller than catheter tip 202. Alternatively, it may be larger. Optionally, catheter 202 has a diameter of between 1 and 5 mm, for example between 2 and 3 mm.

In some exemplary embodiments of the invention, tip 204 includes between 1 and 5 turns, for example, between 2 and 4. Optionally or alternatively, tip 204 has a diameter of between 0.1 and 2 mm, for example, between 0.2 and 1 mm. Optionally or alternatively, tip 204 and a pitch angle of between 10 and 80 degrees, for example, between 30 and 70 degrees.

In some exemplary embodiments of the invention, tip 204 terminates with a sharp tip.

In some exemplary embodiments of the invention, an electrode 206 has an axial extent along tip 204 of between 0.1 and 5 mm, for example, between 0.2 and 2 mm.

In some exemplary embodiments of the invention, a port 208 has an area of between 0.01 and 3 mm².

In some exemplary embodiments of the invention, catheter 202 is made of MR compatible materials, so that the procedure can be carried out, at least in part, using MRI image guidance.

Optionally or alternatively, the helix 204 is formed of metal, for example, stainless steel or Nitinol. Optionally or alternatively, the helix is formed at least in part out of polymer materials, for example, hard plastic, or from glass or from a non-magnetic metal.

### Exemplary GP Injection Method

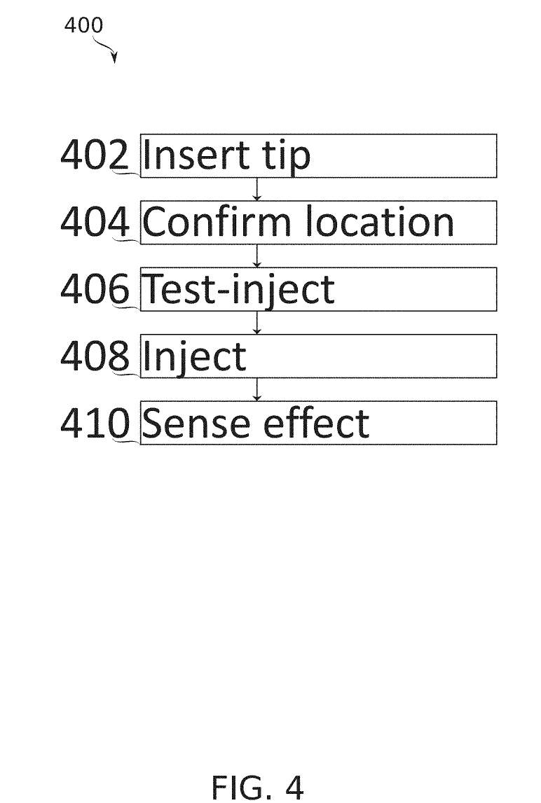

FIG. 4 is a flowchart of a method 400 of injection to a GP, in accordance with some exemplary embodiments of the invention.

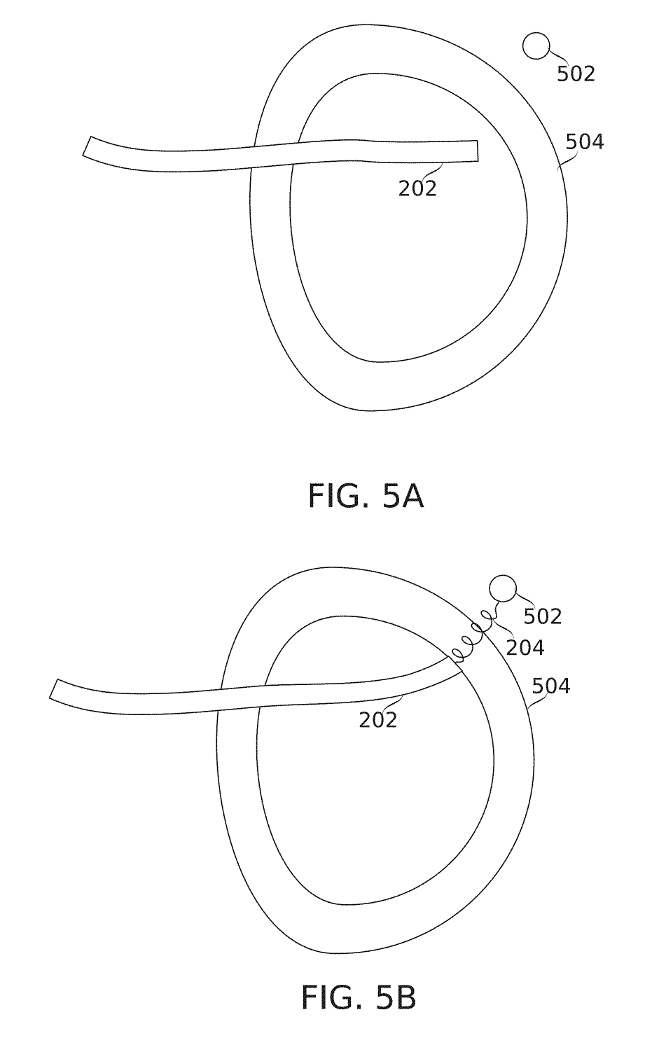

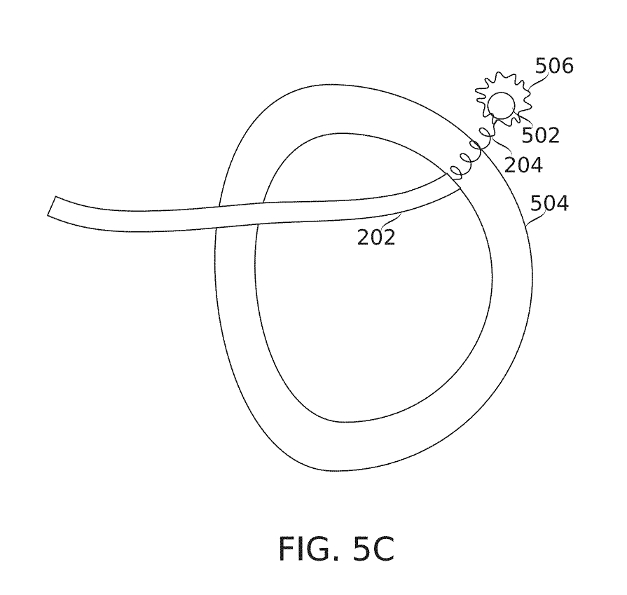

FIGS. 5A-5C are a series of schematics showing stages of a method according to FIGS. 1 and 4, in accordance with some exemplary embodiments of the invention.

FIG. 5A shows catheter 202 near a GP 502, for example, a cardiac GP.

At block 402, tip 204 is inserted into cardiac tissue. FIG. 5B shows insertion of tip 204 into cardiac muscle 504 near GP 502.

At block 404, the location of tip 204 is confirmed, for example, by collecting electrical data or other methods, for example, as described herein.

FIG. 5C shows an optional test injection of contrast material (corresponding to block 406 of FIG. 4), indicating an expected/possible spread of injected treatment material 506.

In some exemplary embodiments of the invention, the injection is gated to local mechanical and/or electrical activity, for example, electrical activity (e.g., ECG or EGG) or mechanical behavior (e.g., using a local strain sensor or accelerometer at the catheter tip). Optionally, this allows injection to be applied when the surrounding muscle is tense or relaxed and/or at a repeated location. Optionally, gating is provided by an injection pump being activated after a user inject-request only when triggered by an ECG input. Optionally, a user elects which part of the ECG (or other signal) acts as a trigger. Optionally or alternatively, the ECG trigger may be used to stop further injection at certain parts of the cardiac cycle.

At block 408, treatment material, for example, viscous botulinum toxin preparation, is injected into a GP for at least temporarily treating it. It is noted that while the effect may not be permanent, a temporary effect of several weeks or months may be sufficient to allow tissue to recover, heal and/or stabilize at a healthier behavior pattern.

In some embodiments, the procedure is, at least in part, under imaging; for example, MRI, CT, NM or ultrasound imaging. Optionally, the injectate includes a marker or contrast material suitable for the imaging method. For example, an injected toxin may include microbubbles or microspheres. Optionally or alternatively, the injectate includes material useful as a reference for later reaching the same location; for example, contrast material. Optionally, the injected material can be used for later modulation, for example, include gold (or other) nanoparticles particles or microspheres for later activation from outside the body, and/or from outside the GP; and/or may include a marker which can be chemically identified if a suitable antibody or other matching chemical is later provided in the body.

In some exemplary embodiments of the invention, ultrasonic monitoring is provided, for example, using intra-cardiac or trans-esophageal echocardiography.

In some exemplary embodiments of the invention, the size of the injected bolus is between 0.5 and 7 mm in maximal extent, for example, between 1 and 5 mm in maximal extent. Optionally, the size of affected area is 1 mm, 2 mm, 3 mm, 4 mm, 5 mm or greater or intermediate in maximal extent. Optionally, these are the sizes of regions to which are provided at least 10% of a maximal dose and/or 50% of an effective dose. In some cases, the treated area may include a tendril, including less than 30%, 20% or 10% of the treated volume, which lies outside the above dimensions.

In some exemplary embodiments of the invention, the injection is of a bolus in the shape of an ellipsoid. Optionally, the injection is into an organelle, such as a GP, and matches its general shape.

In some exemplary embodiments of the invention, for example as described below, a means to reduce injectate migration may be used; for example, suction applied from near-tip region 212.

Optionally, after injection of a treatment material, the tip is optionally moved, and injection of a flushing material, for example, saline or a nullifier of the treatment material (or a material which increases viscosity thereof), may be provided in the vicinity of the GP.

At block 410, an effect of the injection is sensed, for example, in the GP and/or in other nervous tissue and/or by sensing one or more physiological parameter of the organ controlled by the GP or other body system(s).

### Exemplary Alternative Catheter Tip Design

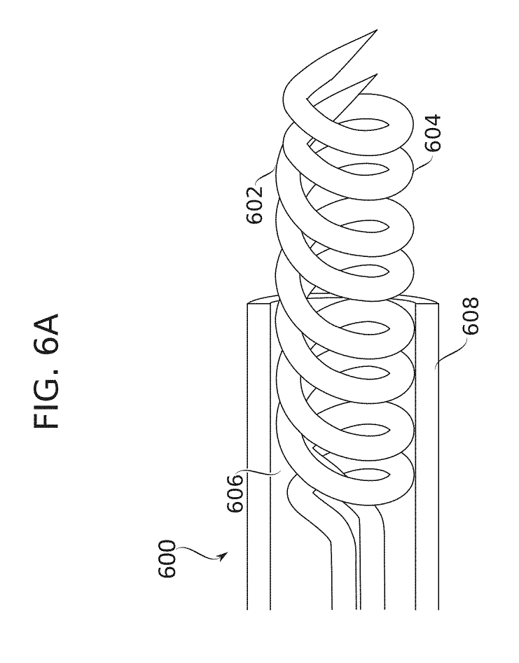

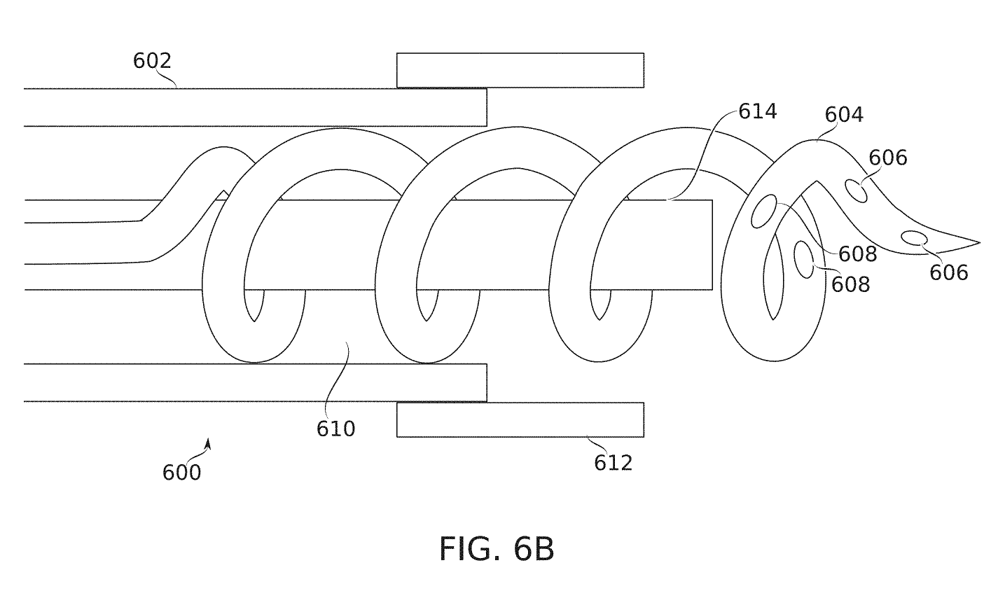

FIGS. 6A-6B show various alternatives for catheter tip design, in accordance with some exemplary embodiments of the invention.

FIG. 6A shows a tip design 600 including a plurality of separate tips 602, 604. In the example shown, both tip 602 and tip 604 are helical. One or both of tips 602 and 604 can include fluid output ports, for example, at their sides or at their ends, for example, as described in relation to FIGS. 2-3 herein. One or both of tips 602, 604 may include one or more electrodes, for example arranged as described in relation to FIGS. 2-3 herein.

In some exemplary embodiments of the invention, each of tips 602 and 604 acts as an electrode, optionally coated with a non-conducting material, exposed at sensing location(s) thereon.

In the embodiment shown, tips 602 and 604 can be advanced out of a lumen 606 in a catheter 608 (e.g., pushed and/or rotated). In some embodiments, one tip is fixed and another is movable. Optionally, a movable tip includes a stop to prevent over-extending. Optionally or alternatively, a tip can be detached from catheter 608, for example, to allow simultaneous implantations in different locations, for example, for simultaneous sensing and/or stimulation and/or treatment.

In some exemplary embodiments of the invention, a tip 602, 604 includes an umbilical cord including, for example, a fluid channel and/or electrical conductor(s).

Optionally, the fluid channels are themselves conducting.

In some exemplary embodiments of the invention, one or both of tips 602, 604 are not helical; for example, being straight. In one example, tip 602 is screwed into tissue (e.g., by rotating catheter 608, optionally being fixed thereto) and tip 604 can then be selectively advanced, for example, within an inner volume defined by such a helical shape, between turns of tip 602.

In some exemplary embodiments of the invention, the use of separate tips allows each tip to be dedicated for injection of a different material (e.g., contrast or treatment), and coupled to a source thereof over the course of a procedure.

In some exemplary embodiments of the invention, two tips 602 and 604 have different mechanical properties, for example, one being longer, one having a larger diameter, one having a larger helix diameter, one having a greater diameter lumen, different port locations and/or different electrode arrangements and/or numbers. In some embodiments, two or more of the above properties are different. Alternatives, the tips may be substantially identical and/or have same values for one, two or more of the above geometric parameters.

While FIG. 6A shows helical needles, in some embodiments helical-shaped tips 602 and 604; in some embodiments a helical tip is terminated with a straight section, for example, axially directed. Optionally, the helical section is used for anchoring in muscle or other tissue, while the straight section is used to minimize mechanical interaction with target tissue. In some embodiments, for example, for ablation, a helical needle may be desired, for example, to allow more even spreading and/or localization of ablation, for example, chemical, heat, electrical or RF type ablation.

Other needle designs may be used. It is noted that a helical design may be useful to reduce backsplash into the lumen/blood.

In some embodiments of the invention, especially for a straight or slightly curved needle, a spring loaded mechanism may be used, in which, when a spring is released, the needle is held into nearby tissue.

In some embodiments, the catheter includes an ultrasound imager and/or an ultrasonic depth sensor. Optionally, a separate ultrasonic imager is provided.

In some exemplary embodiments of the invention, the catheter includes navigational means, for example, a guide wire, bendable tip and/or position sensor. In some embodiments, such functions are provided in a separate element, with which the catheter can be combined, for example, a guide sheath.

Optionally, for example for the heart, access is not from inside a chamber. For example, the catheter may be used to exit a cardiac vein (e.g., using the needle tip or other tip to form a hole therein and extend the catheter through the hole into a pericardial space). The catheter may then be navigated to near a GP or other tissue to be treated.

In some embodiments, injection is into a blood vessel feeding GP or tissue to be treated. Optionally, the injected material includes particles large enough and/or is viscous enough so that it does not pass through capillaries.

FIG. 6B shows an alternative tip design 600, in accordance with some embodiments of the invention. A catheter 602, optionally a polymer-coated braid or coil, includes a lumen 610. Optionally, lumen 610 is used for delivering contrast material; for example, for use in navigation of catheter 602. Optionally, a helix 604 is provided at an end of catheter 602, for example, a helix which is selectively advanceable out of lumen 610. Optionally, helix 604 includes one or more electrodes 606 and/or one or more fluid ports 608, which may be used, for example, as described, e.g., in relation to FIGS. 2-3 herein. Optionally, helix 604 itself acts as an electrode and catheter 602, for example at its tip (which is optionally exposed to fluid) acts as a second electrode. A cover 612, optionally a retractable overtube or a fixed layer, acts to define a tip of catheter 602 and/or selectively cover helix 604. Optionally, cover 612 is electrically conducting and acts as an electrode.

In some embodiments, an electrically conducting cover 612 can be used for ablation when the helix is retracted, for example, using a monopolar method or bipolar methods (e.g., if cover or tip includes two electrodes, or if tip of helix acts as a second electrode).

In some exemplary embodiments of the invention, a stylet 614 is provided in lumen 610, optionally radially within helix 604. Optionally, stylet 614 is used for controlling depth of penetration of the helix and/or for controlling a shape of catheter 602, for example, for navigation. Optionally or alternatively to a stylet, a guide wire is provided.

### Exemplary Catheter Tip Fluid Pathways

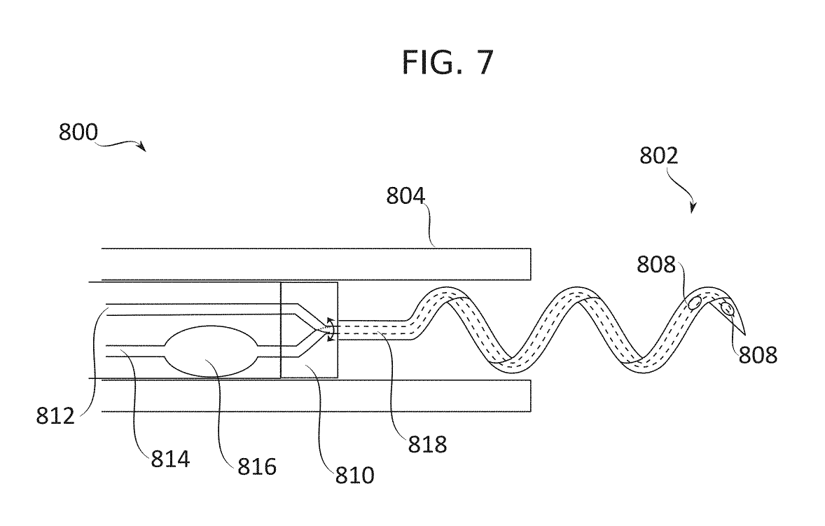

FIG. 7 illustrates fluid flow in a catheter tip, in accordance with some exemplary embodiments of the invention.

In some embodiments of the invention, relatively viscous materials are injected into a GP. It may be desirable to maximize a flow channel diameter for them. In some embodiments, a catheter is used to inject both contrast material and a treatment material (e.g., viscous). In some embodiments, multiple channels 208′ extend the entire length of the catheter 202. However, even if the viscous material channel is allocated a greater cross-section, there may be an undesirable amount of resistance to fluid flow. Furthermore, the diameter of the tip generally further increases resistance to flow of viscous materials and may present a greater difficulty.

FIG. 7 shows a design 800 for a catheter 804 in which a single tip channel 818 is shared by two separate fluid source channels 812, 814. A tip 802, for example a helical tip, for example, having two (or more) side output ports 808 has a single lumen 818 along its length.

In some exemplary embodiments of the invention, a no-backflow valve 810 is optionally provided at a junction of channels 812 and 814. If pressure is increased in one of channels 812 and 814 the material in that channel flows forward to tip channel 818 and out of ports 808. Optional valve 810 prevents contamination of the other channel. More than two channels 812 and 814 may be provided. Separate anti-backflow valves may be provided; for example, one per channel. In some embodiments, valve 810 is an active valve, for example, controlled electrically, and can selectively open and link one of channels 812 or 814 to tip channel 818.

In some exemplary embodiments of the invention, after injection of contrast material, treatment material is injected, which first flushes out any material in channel 818. Optionally, flushing of channel 818 is performed after injection of treatment material, for example, using saline (in the contrast channel) or contrast material. Such flushing and injection may also be used in embodiments where there is only a single channel 812, 814. Optionally, for example to deal with high viscosity of the treatment material, a reservoir 816 is provided near tip 802 and from which the travel distance to ports 808 is short. Optionally, reservoir 816 includes enough material for treating 0.5, 1, 2 or an intermediate or greater numbers of GPs. Optionally, a lower viscosity material is used to flush higher-viscosity material through the channels.

Optionally, the treatment material is binary and is provided via two separate channels or through a dedicated channel and the contrast material channel.

### Exemplary Catheter Tip Depth Sensing

In some exemplary embodiments of the invention, there is providing a depth sensing mechanism, for example, to determine a depth of tip 204 in the tissue and/or to ensure that tip 204 does not penetrate into, for example, a lumen or tissue which is not target tissue. In the heart, it may be desirable to avoid penetrating to a pericardial space. In some embodiments, impedance sensing (e.g., between electrodes 206) is used to identify the tissue at tip 204.

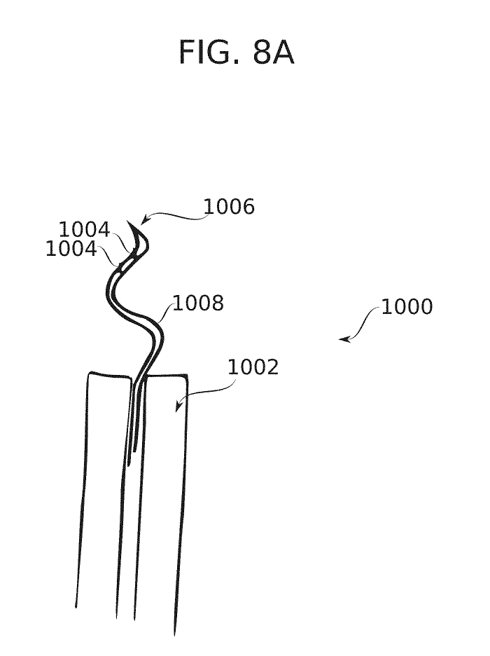

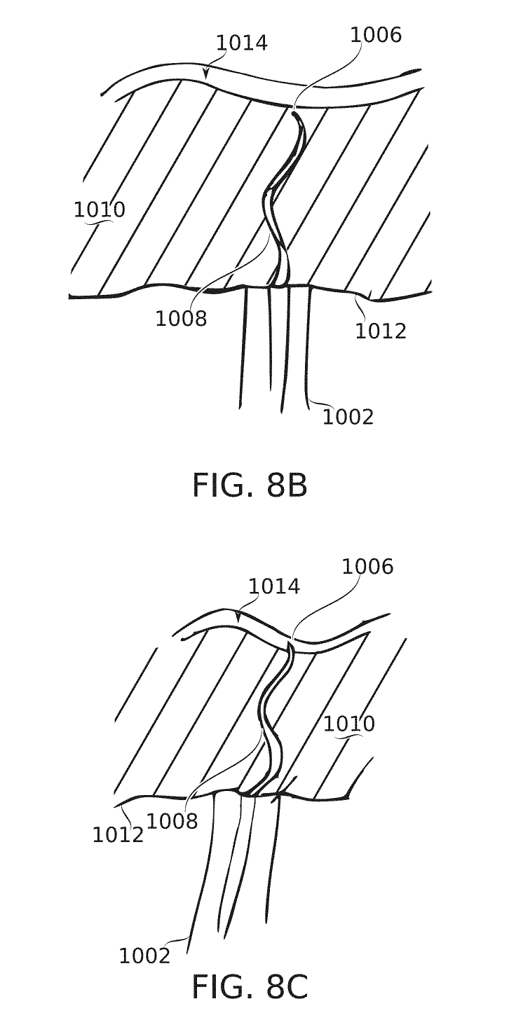

FIGS. 8A-8C show the use of an optical sensor to avoid over-penetrating through a wall into a lumen. Such penetration may be undesirable as it may allow any injected material to travel far away from a GP to be treated.

FIG. 8A shows a catheter tip 1002 of a catheter 1000, in accordance with some embodiments of the invention. A plurality of irrigation ports 1004 are shown on a helical part 1008 of the tip, as an example of a treatment mechanism whose positioning relative to a GP it is desired to control. In an exemplary embodiment of the invention, an optical sensor 1006 (e.g., an optical transceiver), for example an optical fiber, optionally threaded in a lumen which can be used to carry fluid to ports 1004, is shown.

FIG. 8B shows a catheter tip 1002 brought into contact with a myocardial surface 1012 and helical part 1008 advanced into cardiac tissue 1010. In FIG. 8B, sensor 1006 is within tissue 1010, so a degree of reflection of light from sensor 1006 and back to sensor 1006 is, for example, low. In FIG. 8C, sensor 1006 has been advanced into a pericardial space 1014 and therefore a reflection is different, for example, high. Optionally, a light source and sensor are provided outside the body and attached to sensor 1006 by fiber optics. Alternatively, a LED source and/or electro-optic transducer are provided at the location of sensor 1006.

In an alternative design, sensor 1006 is advanceable ahead of tip 1008 and can thus provide an indication before fluid ports 1004 enter a pericardial space.

### Retention of Injected Material

In some exemplary embodiments of the invention, the injected material is toxic and/or can have undesirable effects if it travels in an undiluted form far from its target area (e.g., a GP or other nervous tissue and/or other tissue to be treated). In some embodiments the injected material is viscous and/or sets in contact with tissue and/or is mixed on the spot from two components, so that it sets.

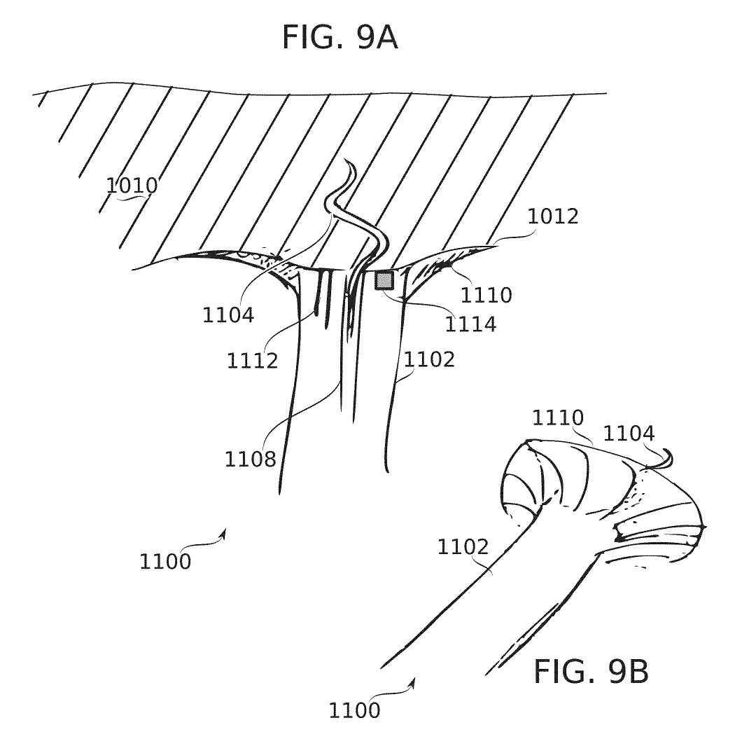

FIGS. 9A-9B show a catheter 1100 with a fluid retention mechanism that reduces migration of injected material, in some exemplary embodiments of the invention.

In the embodiment shown, a catheter tip 1102 includes an optional skirt 1110 and one or more suction outlets 1112. In use, for example, before, during and/or after injection of a fluid via an aperture in a helical tip 1104 (or other means), the application of suction encourages material prone to migration, to migrate towards suction port 1112 (e.g., and to an optional suction source outside the body), rather than away from tip 1102. In some exemplary embodiments of the invention, skirt 1110 is flexible and has a radial extent of, for example, between 1 and 5 mm, for example, about 3 mm away from the catheter perimeter. Optionally, port 1112 is in the form of a plurality of ports and/or a ring of ports or an annular port. Optionally, suction is applied, for example, for between 1 and 200 seconds after injection, for example, less than 30 seconds.

In some exemplary embodiments of the invention, the injected material is not viscous and a polymerization aid is used to help it set quickly. Optionally, the polymerization aid is the injection of another chemical. Optionally or alternatively, a UV light source 1114, for example, an optical fiber connected to a source outside the body, is used to induce local polymerization.

Optionally or alternatively, flushing is used to dilute any migrating material.

Optionally, flushing is applied from fluid ports that are not in the GP, for example, more distal and/or more proximal than those used for treatment.

### Exemplary Depth Control Mechanism

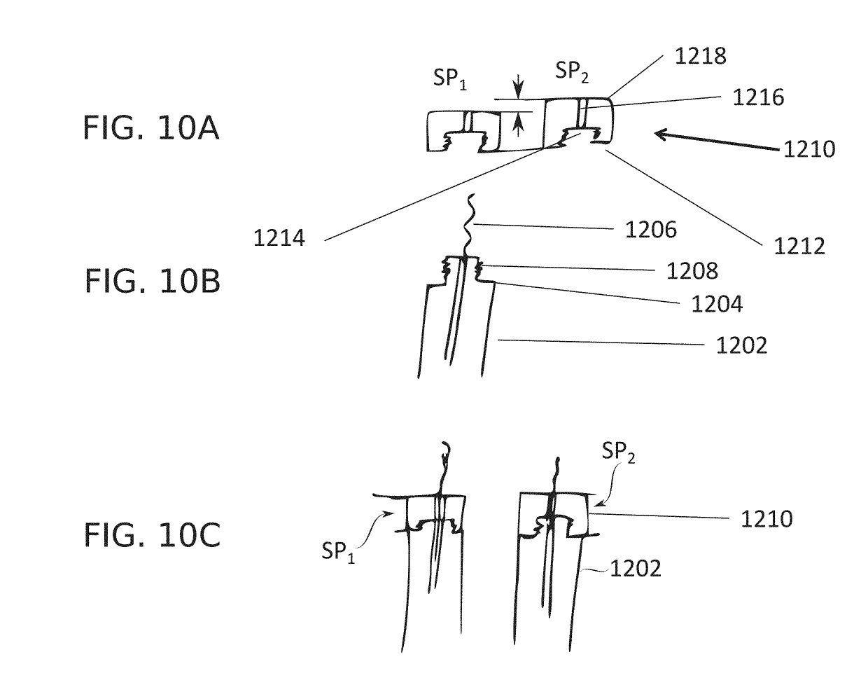

FIGS. 10A-10C show a depth control mechanism for a catheter tip 1202, in accordance with some exemplary embodiments of the invention.

In some exemplary embodiments of the invention, the mechanism is in the form of a cap 1210 which is attached to the distal end of tip 1202 and thus effectively shortens a distance between a distal end of a helical needle 1206 and a near-tip part 1204 of catheter tip 1202.

In some exemplary embodiments of the invention, the distance between an inner wall of a lumen and a GP is predetermined, for example, using imaging (e.g., CT) or anatomical considerations and a cap 1210 having a desired offset (e.g., distance between a proximal face 1212 and a distal face 1218) is selected. Optionally, a kit is provided with several (e.g., 2, 3, 4, 5 or more) sizes of caps 1210 (e.g., spanning range of 1-5 mm, for example, in 1 mm steps). Optionally or alternatively, a single cap is cut down to size, as needed.

In one example, cap 1210 includes an inner threading 1214 or other interference based geometry which interlocks with a matching geometry 1208 on the tip of catheter tip 1210. Optionally, a channel 1216 for helical needle 1206 is provided in the cap. Optionally, the channel is in the form of a slot.

In an alternative embodiment, different helical needles are used, according to a desired length thereof.

In an alternative embodiment, a tube (not shown) is advanced over catheter tip 1210 or within helical needle 1206 to prevent over advancement thereof.

### Exemplary Signal Sensing and Processing

In some exemplary embodiments of the invention, sensing is focused on sensing electrical activity in the GP. In some exemplary embodiments of the invention, sensing is during modulation, for example, to measure a response of the GP and/or others parts of the ANS to an injected material (in the GP and/or other parts of the ANS) and/or to assess an efficacy of such injection.

In some exemplary embodiments of the invention, however, electrical sensing is provided for other than merely within-GP activity.

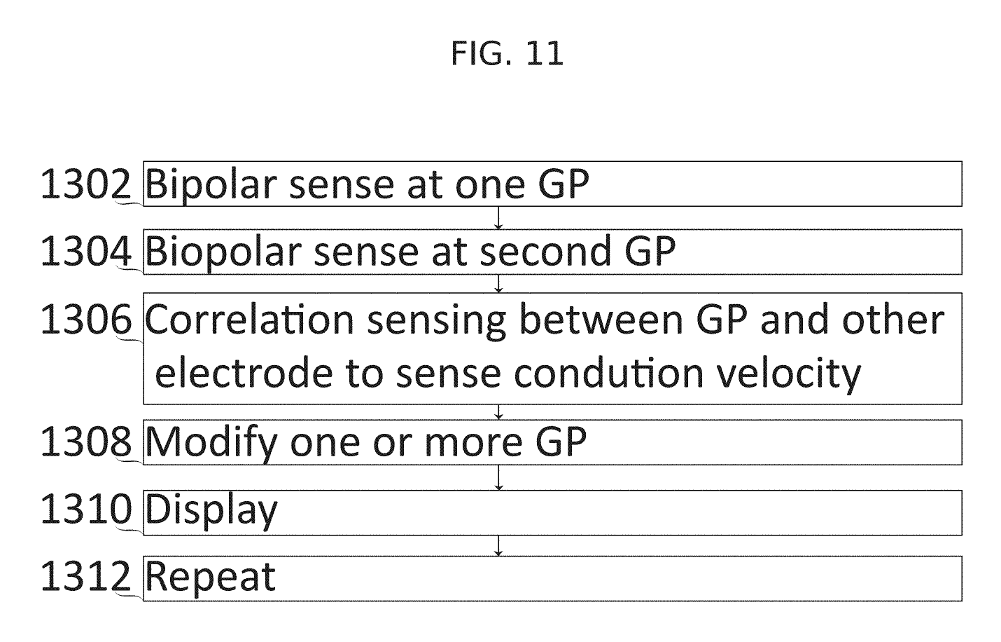

FIG. 11 is a flowchart of a method of signal sensing and/or processing, in accordance with some exemplary embodiments of the invention.

At block 1302, bipolar sensing of activity within one GP is performed.

At block 1304, bipolar sensing within another GP is optionally performed.

At block 1306 a correlation between two GP sensings and/or a GP activity and other electrical or other measurements (e.g., contraction force) are made.

At block 1308 one or more GPs and/or other physiological parameter are optionally modified, for example, by electrical stimulation.

At block 1310, previously and/or currently collected data is optionally displayed.

Optionally, the display is an overlay on a previously prepared anatomical map and/or map of GP network. Optionally, position sensing of the tip of the catheter is used to help register sensed data to a previous set of data.

Optionally, such a display is used to monitor the effect of local injection (e.g., of botulinum toxin), for example, to allow comparison of before and after. Optionally, this is used to validate that injection (or ablation) was to the correct place and/or that it had a desired effect. This may allow correction during the procedure.

At block 1312, any or all of acts of blocks 1302-1310 are repeated.

Electrical and/or other sensing can be used to indicate, for example, tissue state, reactivity and/or activity.

In one example, endocardial electrograms are measured to reflect autonomic effect (e.g., of GP manipulation. For example, such measurement can include measuring local conduction velocity and/or direction. This may be useful during irregular rates such as flutter and fibrillation. Optionally or alternatively, local conduction speed is measured, for example, using a multi electrode catheter and a pacing probe on same catheter. Optionally or alternatively, action potential duration and/or other parameters are measured, for example, using methods known in the art, including bipolar and/or unipolar electrodes.

In another example, correlations between GP activities and GP changes and cardiac electrical parameter changes, are tracked.

In a particular example, conduction from a GP to another location in the heart is measured.

In another example, neural data is measured from the skin surface, for example, using the methods described in U.S. Pat. No. 8,744,571, the disclosures of which are incorporated herein by reference.

Optionally or alternatively to electrical measurements, other physiological measurements may be taken, for example, heart rate, contractility, local contraction force, local tissue movement, local oxygenation, local blood flow and/or stroke volume. In some exemplary embodiments of the invention, such measurements are used during intervention (e.g., GP or organ modulation). Optionally, before, during and/or after measurements are shown super imposed and/or automatically analyzed, for example, to show transient, possibly minute, changes.

In one example, a signal S, is recorded for period t that is smaller or equal to the average cardiac cycle, T, of a patient. Then, several consecutive signals (optionally with some beats ignored) are averaged to create an average signal SA.

Optionally, binning is used so that signals from similar beats are combined.

In some exemplary embodiments of the invention, SA, is continuously updated.

In some exemplary embodiments of the invention, a real time depiction of S-SA is provided. This may enable an operator to monitor minute changes in S of a single beat or a small number (e.g., fewer than 30 beats, averaged, for example).

Optionally, the largest change recorded is called the Delta Change.

In some exemplary embodiments of the invention, an intervention to record the effect of the autonomic modulation will be tested by one or more of:
Invoking a short term change in autonomic tone by inducing, for example, one or more of:
   i. PVC
   ii. APC
   iii. Abrupt Mechanical load change (e.g., instantaneous injection)
   iv. Other temporary intervention, for example:
Recording the Delta Change that is related to the intervention.
Recording the change of Delta Change before during and after an autonomic modulation therapy.

### Exemplary Injected Materials

In an exemplary embodiment of the invention, the injected material has a temporary effect on nervous (or other target) tissue, for example, 1-10 days, 1-10 weeks or 1-10 months. In an exemplary embodiment of the invention, the material includes Botulinum toxin A. (Xeomin, Merz Pharma GmbH & Co KGaA, Germany; e.g., 50 U/l mL at each GP). Optionally, the amount injected depends on the size of the bolus. Optionally, the type of toxin is selected according to a desired duration of effect. Optionally, a mixture of different toxins (or different injections) are used to provide a first magnitude of effect for a first time and a second magnitude of effect for a second time. Optionally, different GPs are injected with different toxins which have different effects and/or different duration of effects. For example, Botulism toxin A with an effect of between a few days and a few weeks or months can be selected based on the strain of the bacteria making the toxin.

In some exemplary embodiments of the invention, the volume injected is equal to between 5% and 200% of a desired bolus volume and/or affected volume size, for example, between 10% and 30%.

In some embodiments, the injected material (e.g., a fast acting analgesic material) has a short enough effect that it can pass during a procedure, e.g., within fewer than 30 minutes, 20 minutes, 10 minutes, 3 minutes or less.

In some embodiments, the injected material causes a permanent ablation, which may be overcome by tissue regrowth and/or plasticity, for example, if alcohol is injected (e.g., Ethanol 99.6%, 0.5-1 ml).

In some exemplary embodiments of the invention, the injected material is selected to be viscous up to the viscosity limit of injection catheters, for example, by adding hyaluronic acid, carbomer, polyacrylic acid, cellulose polycarbophil, polyvinylpyrrolidone, gelatin, dextrin, polysaccharide, polyacrylamide, polyvinyl alcohol, polyvinyl acetate, chitosan, algenates and derivatives and mixtures.

Optionally, this reduces the chance of unintended migration. (e.g., at a viscosity of viscosity 3-100 cP, for example, 5-50 cP, for example, 10-50 cP, for example, 20-45 cP).

In some exemplary embodiments of the invention, an adjunct is injected, for example, to reduce local primary inflammation attributed to cytokines produced by local atrial pericardial fat and/or reduce secondary inflammation induced as local adverse reaction of the tissue to the injected material and/or act of injecting and/or nearby ablation(s). In one example, an anti-inflammatory steroid, optionally a NSA, is injected to reduce an inflammatory reaction, e.g., Triamcinolone acetate 1 mg/kg.

Optionally, the injected material is a mixture of up to 10% or 30% toxin, up to 10% or 30% thickener and up to 10% or 30% anti-inflammatory. Optionally, at least 0.01% of each component is provided. For some toxins, the actual amount will be far lower, for example, less than 0.01%

### Exemplary Complete Process

Following is a description of a complete treatment process for, for example, AF (atrial fibrillation), optionally using the apparatus and/or methods as described herein, in accordance with exemplary embodiments of the invention. It should be appreciated that in an actual procedure, one or more of the acts described below may be omitted and/or order changed. Optionally, the methods and/or apparatus of U.S. Provisional Filing No. 62/160,080; filed May 12, 2015 are used.

First, a patient health record is optionally viewed and analyzed to determine possible underlying conditions and/or symptoms.

Next, pre-acquired data (e.g., NM, CT, ultrasound and/or MRI) are analyzed. In one example, such data is segmented, positions and/or sizes of various anatomical and/or functional features are extracted, data, such as blood flow, electrical activity, dielectric stimulation results are added. In particular, MR data is optionally analyzed to determine atrial wall thickness near GPs. Optionally or alternatively, MR data is analyzed to generate a measure of fibrosis (e.g., using Gd-DE, such as a Utah score). NM data is optionally analyzed to extract MIBG and MIBI data, for example, to detect the degree of activity and/or location of GPs and/or regional distribution of perfusion/innervation match and/or mismatch.

Optionally, data from multiple imaging modalities are co-registered and overlaid.

Optionally, pre-planning of the procedure is carried out, for example, using the data.

Optionally, such pre-planning includes one or more of indicating function of tissue, marking landmarks and/or tissue to be treated and/or tissue to be avoided (e.g., nerves). Optionally or alternatively, planning comprises planning a trajectory for a catheter and/or planning pathways to be modulated and/or planning GP locations to be modulated.

Optionally, after planning, simulation may be carried out. Optionally, simulation uses a 3-D mesh model conduction of the tissue to assess effect of modulation.

Optionally or alternatively, simulation may include estimating the effect of an error and/or generating one or more signals that are expected to be measured during and/or after the procedure, under one or more conditions. Optionally, a success rate is estimated/calculated based on the plan and the model.

In some exemplary embodiments of the invention, the procedure is carried out using a single catheter (e.g., as shown in FIG. 3 or 6A or 6B), which has a handle connected to an ablation and/or modulation system (e.g., a standard system as used for PVI) and optionally, for example, via a separate connector to a dedicated system (e.g., for GP sensing, ablation and/or modulation).

First, the catheter is inserted into the body (e.g., via a vein) and navigated to the target organ (e.g., the heart).

The catheter location is optionally registered to the planning data and/or pre-acquired data, for example, using methods known in the art.

The catheter is pushed through the septum to the left atrium.

A PVI procedure may be carried out, for example, using the tip, optionally with the helix retracted or covered. Alternatively, the catheter used for PVI is different than the one used for GP modulation.

In some embodiments, a dedicated PVI catheter, e.g., using a balloon for positioning, is used.

In some exemplary embodiments of the invention, feedback on the ablation and/or modulation process is used and/or shown to a user, for example, temperature feedback or impedance feedback (e.g., to indicate contact pressure and/or change in tissue). For example, ablation at 25 W, to reach a temperature (measured, e.g., using a temperature sensor at a near-tip region) of 80° Celsius and an impedance of 60 ohm.

After PVI (if carried out), one or more GPs are optionally treated. Optionally, the planning is used to guide the catheter to a GP location and then this location is verified, for example, using a HFS, for example, 20 Hz, 5 ms, 15 mA, optionally using the ablation and//or modulation catheter and/or a helix tip as described above (optionally with minimal or no penetration). A change in heart rate is expected if the local stimulation hits a GP.

Individual GPs may be treated, for example, using modulation as described above or by deep ablation from the epicardium. A more complex process of temporary modulation, verification, ablation and/or determination of effect on body may be carried out, for example, as described herein.

Optionally, ablation of a GP is validated, for example, using HFS.

Optionally, the procedure is validated, for example, by passing a multi-electrode catheter, such as a ring catheter and determining if ablation blocks conduction between separate electrodes of the catheter.

Re-ablation for PVI or for GP modulation may be carried out, if needed.

### GP Modulation

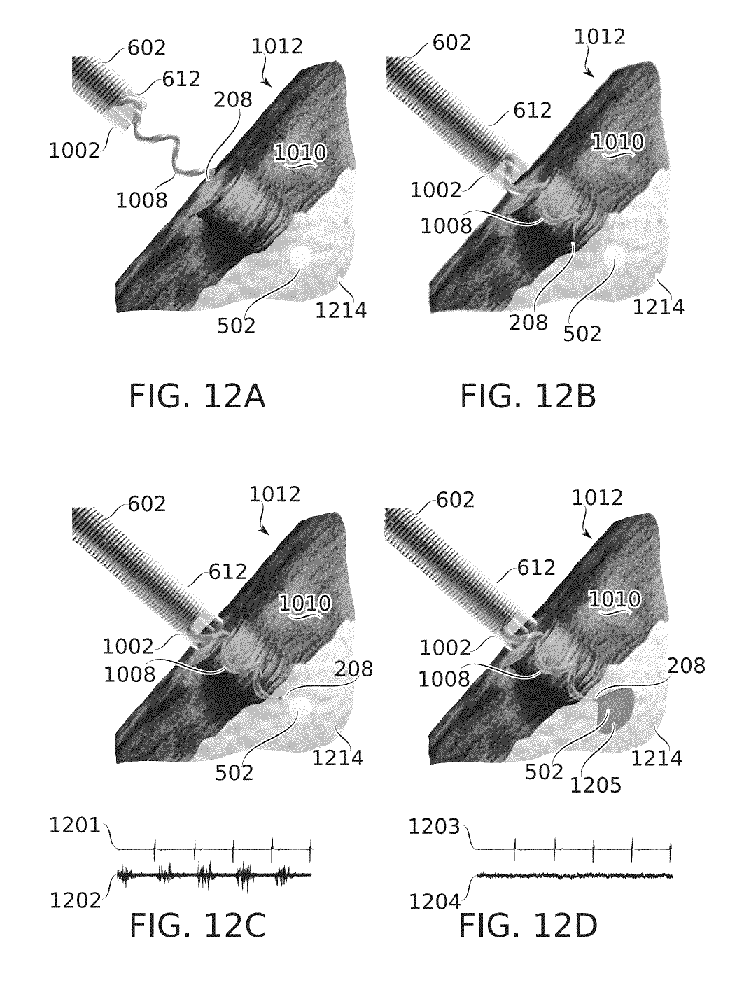

Reference is now made to FIGS. 12A-12D, which schematically represent a sequence of operations for injection to a GP, according to some embodiments of the invention.

FIG. 12A, in some embodiments, shows helical tip 1008 protruding from distal end 1002 of catheter 602, and in contact with myocardial surface 1012 of cardiac tissue 1010. Cover 612 extends distally a short distance from the body of the catheter 602; optionally, the remaining distance from the distal end of cover 612 to the distal end of helical tip 1008 defines a maximum penetration distance of helical tip 1008. Targeted GP 502 is shown embedded in fat pad 1214.

FIG. 12B, in some embodiments, shows helical tip 1008 penetrating into the cardiac tissue 1010. In FIG. 12C, penetration has advanced until fluid port 208 is within injection range of target GP 502. ECG trace 1201 shows heart activity accompanying ganglionic activity shown in ENG 1202.

At FIG. 12D, in some embodiments, a neural activity blocker 1205 (e.g., botulinum toxin or cold saline) has been injected. ENG 1204 now shows substantially reduced activity, while ECG activity continues. Reduction in ENG activity may partial to complete; reduction in ENG activity may be immediate upon injection and/or reducing over time. In some embodiments, injection is performed gradually under the control of feedback from analysis of the ENG activity decrease. Optionally, failure to achieve a certain expected partial level of blockage during a certain portion of an injection protocol is used as a basis for adjusting a position of the helical tip 1008 to a position which potentially delivers activity blocker 1205 more accurately to the target.

### Imaging-Guided GP Ablation

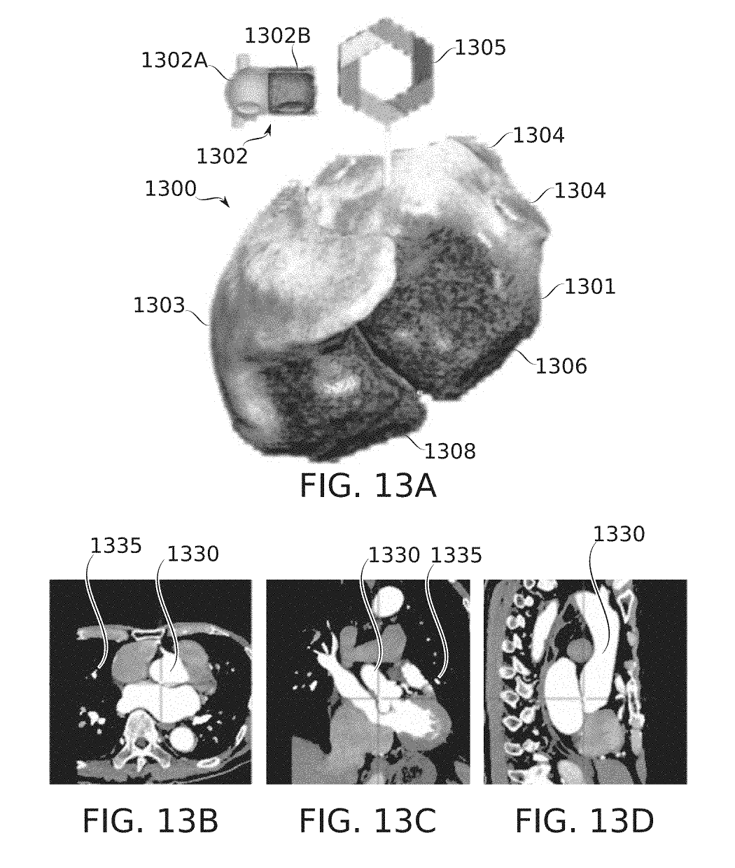

Reference is now made to FIG. 13A, which represents a 3-D view of the atria 1300 of a heart reconstructed from CT data, according to some exemplary embodiments of the invention. Heart orientation icon 1302 shows the relative orientation of the left atrium 1301, represented by icon section 1302B and the right atrium 1303, represented by icon section 1302A in the view shown. Orientation tag 1305 is rendered as a “lollipop” marker, oriented so it presents a flat surface to a dorsal or ventral view, and an edge-on appearance to lateral views.

In the figure, display of the ventricles is suppressed; valves leading from the atria are shown at 1306 and 1308. Two pulmonary vein roots of the left atrium 1301 are visible at 1304.

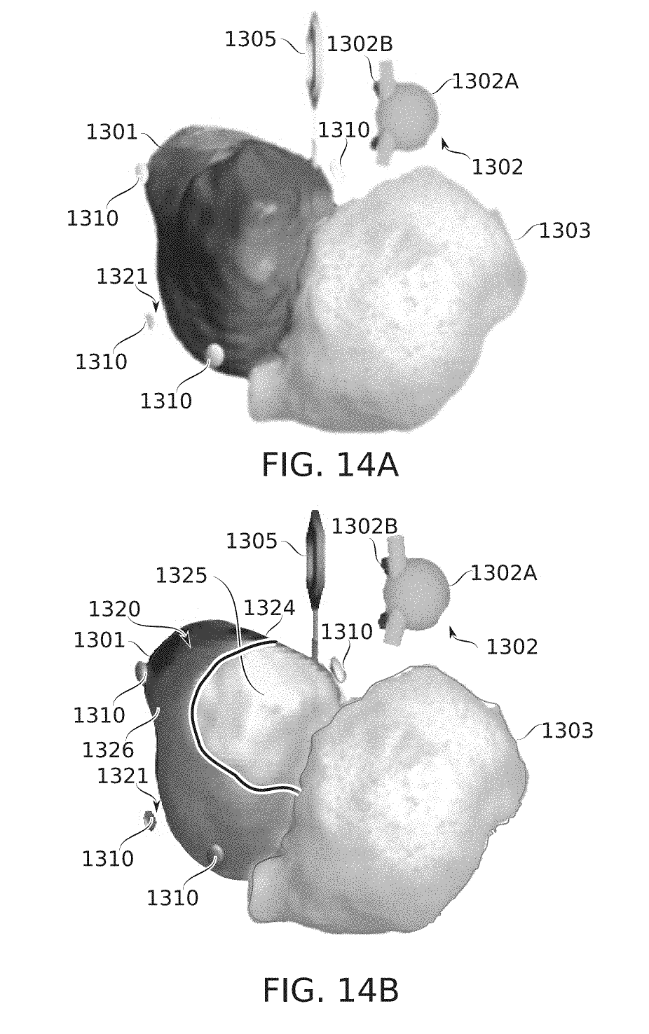

Reference is also made to FIGS. 13B-13D, which show ¹²³I-mIBG NM images in the transverse, frontal (coronal), and medial (sagittal) planes, respectively, the NM images being fused with CT data, according to some exemplary embodiments of the invention. In some embodiments, ¹²³I-mIBG imaging is used to highlight relative differences in autonomic innervation of the heart (¹²³I-mIBG is chemically similar to norepinephrine, which is involved in autonomic regulation of the heart). Relative uptake is determined, for example, based on heart/mediastinal ratio (H/M) of image intensity, trace washout rate, and/or focal uptake defects. Brighter areas potentially reflect areas of greater nerve activity, including innervated cardiac tissue 1330 and ganglia 1335. Reference is now made to FIGS. 14A-14B, which show atria 1301 and 1303 in a lateral view, together with ganglia 1310, according to some exemplary embodiments of the invention. In FIG. 14A-B, right atrium 1303 is shown with lowered contrast to emphasize features of the display of left atrium 1301.

Ganglia 1310 surrounding left atrium 1301 are represented as “floating” off the surface of the atrium, separated from it by pericardial space 1321. Heart orientation icon 1302 again shows the relative orientation of the left atrium 1301, represented by icon section 1302B; and the right atrium 1303, represented by icon section 1302A in the view shown. The lateral view is also indicated by the sideways orientation of orientation tag 1305.

Left atrium 1301 is shaded in FIGS. 14A and 14B to represent relative uptake of ¹²³I-mIBG (represented by the heart/mediastinum uptake ratio at each shaded region; darker areas represent relatively larger uptake) in baseline conditions (FIG. 14A), and immediately after inactivation of ganglia 1310 (FIG. 14B), for example after inactivation according to the injection method of FIGS. 12A-12D. The inactivation substance used was botulinum toxin.

Brighter areas, particularly the area 1325 within contour 1324 of FIG. 14B, reflect lower ¹²³I-mIBG uptake associated with lower neural activity. Darker areas (e.g. region 1326, and the more uniformly dark appearance of the left atrium 1301 in FIG. 14A) represent relatively higher uptake. Contour 1324 is drawn at approximately the level of H/M=1; thus, at least region 1325 appears to be substantially denervated.

### General

It is expected that during the life of a patent maturing from this application many relevant injection mechanisms will be developed; the scope of the term fluid port is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

1. A method of sensing the activity of nervous tissue of a patient, comprising:
(a) advancing a part of a catheter including an electrode into nervous tissue of the Autonomous Nervous System (ANS) to be sensed;
(b) modulating the activity of the nervous tissue while the catheter is in the body of said patient; and
(c) sensing activity of the nervous tissue using the electrode before, during and/or after the modulating.

2. The method according to claim 1, wherein the nervous tissue comprises a ganglionic plexus (GP).

3. The method according to claim 1, wherein the sensing comprises sensing before and sensing after the modulating.

4. The method according to claim 1, wherein modulating comprises electrical stimulation.

5. The method according to claim 1, wherein modulating comprises modulating an activity of the nervous tissue with an effect expected to last at least 3 weeks.

6. The method according to claim 5, wherein the modulating comprises ablating.

7. The method according to claim 6, wherein ablating comprises ablating using the electrode.

8. The method according to claim 5, wherein the modulating comprises injecting a chemical composition into the nervous tissue.

9. The method according to claim 8, wherein the chemical composition comprises botulism toxin.

10. The method according to claim 8, wherein the chemical composition comprises an anti-inflammatory.

11. The method according to claim 8, wherein the chemical composition comprises a thickener.

12. The method according to claim 8, comprising a first injection to determine an expected spread of the chemical composition.

13. The method according to claim 8, comprising applying suction during or after the injection to reduce spreading of the chemical composition away from the nervous tissue.

14. The method according to claim 8, using a same fluid port for the chemical composition and for injecting a different material.

15. The method according to claim 8, using a different fluid port for the chemical composition and for injecting a different material.

16. The method according to claim 6, comprising also performing pulmonary vein isolation using a same catheter as said catheter.

17. The method according to claim 1, comprising selecting a spacer which sets a penetration depth of said part and attaching the spacer to a distal end of the catheter.

18. The method according to claim 17, wherein the selecting comprises selecting based on an image of tissue in the vicinity of the nervous tissue.

19. The method according to claim 1, wherein said part extends from a distal end of the catheter and the distal end does not penetrate the nervous tissue.

20. The method according to claim 19, wherein said part is helical.

21-50. (canceled)
